(19) United States
(12) Patent Application Publication
(10) Pub. No.: US 2013/0034841 A1
(43) Pub. Date: Feb. 7, 2013

(54) SYSTEMS AND METHODS FOR ISOLATING AND CHARACTERIZING TARGET MATERIALS OF A SUSPENSION

(71) Applicants: Ronald C. Seubert, Sammamish, WA (US); Jackie L. Stilwell, Sammamish, WA (US); Paul Goodwin, Shoreline, WA (US); Martha Stone, Woodinville, WA (US); Arturo Bernardo Ramirez, Seattle, WA (US)

(72) Inventors: Ronald C. Seubert, Sammamish, WA (US); Jackie L. Stilwell, Sammamish, WA (US); Paul Goodwin, Shoreline, WA (US); Martha Stone, Woodinville, WA (US); Arturo Bernardo Ramirez, Seattle, WA (US)

(21) Appl. No.: 13/565,104

(22) Filed: Aug. 2, 2012

Related U.S. Application Data
(63) Continuation-in-part of application No. 13/437,616, filed on Apr. 2, 2012.
(60) Provisional application No. 61/514,102, filed on Aug. 2, 2011.
(60) Provisional application No. 61/577,866, filed on Dec. 20, 2011.

(51) Int. Cl. C12Q 1/04; C12M 1/42; C12M 1/34; C12Q 1/70

(52) U.S. Cl. 435/2; 435/34; 435/5; 435/287.9; 435/288.1

(57) ABSTRACT

Systems and methods for isolating and characterizing various target materials of a suspension are disclosed. A suspension suspected of containing the target materials is added to a tube. A float with a specific gravity corresponding to that of the target material is inserted into the tube. The tube, float, and suspension are centrifuged together causing the various materials suspended in the suspension to separate into different layers along the axial length of the tube according to their specific gravities. The float and/or tube are configured to drive the various target materials to a region of space between the float and inner wall of the tube.

### CROSS-REFERENCE TO RELATED APPLICATIONS

This application claims the benefit of Provisional Application No. 61/514,102, filed Aug. 2, 2011, and is a continuation-in-part of application Ser. No. 13/437,616, filed Apr. 2, 2012, which claims the benefit of Provisional Application No. 61/577,866, filed Dec. 20, 2011.

### TECHNICAL FIELD

This disclosure relates to capturing and isolating target materials of a suspension.

### BACKGROUND

Suspensions often include particles of interests that are difficult to isolate and characterize because the particles occur with such low frequency. For example, blood is a suspension of various particles that is routinely examined for the presence of abnormal organisms or cells, such as circulating tumor cells (“CTCs”), fetal cells, parasites, microorganisms, and inflammatory cells. CTCs are of particular interest because CTCs are cancer cells that have detached from a primary tumor, circulate in the bloodstream, and may be regarded as seeds for subsequent growth of additional tumors (i.e., metastasis) in different tissues. As a result, detecting, enumerating, and characterizing CTCs may provide valuable information in monitoring and treating cancer patients. Although detecting CTCs may help clinicians and cancer researchers predict a patient's chances of survival and/or monitor a patient's response to cancer therapy, CTC numbers are typically very small and are not easily detected. In particular, typical CTCs are found in frequencies on the order of 1-10 CTCs per milliliter sample of whole blood obtained from patients with a metastatic disease. By contrast, a single milliliter sample of whole blood typically contains several million white blood cells and several billion red blood cells.

However, characterizing a particular type of low frequency particle of interest can be difficult when the suspension includes other particles of similar shape, size, and density. For example, characterizing CTCs in a blood sample can be difficult because a typical blood sample includes other cells with similar shape, size, and density such as white blood cells, and may include more than one type of CTC. Practitioners, researchers, and those working with suspensions continue to seek systems and methods for isolating and characterizing particles of the suspension.

### SUMMARY

This disclosure is directed to systems and methods for isolating and characterizing various target materials. A suspension suspected of containing a target material is added to a tube. A float is also added to the tube containing the suspension. The float has a specific gravity that positions the float at approximately the same level as a layer containing the target materials when the tube, float and suspension are centrifuged. The tube, float, and suspension are centrifuged together causing the various materials suspended in the suspension to separate into different layers along the axial length of the tube according to their specific gravities. The float and/or tube are configured to attach or attract the various target materials to the main body of the tube so that the target materials can be isolated and characterized.

### BRIEF DESCRIPTION OF THE DRAWINGS

FIGS. 1A-1B show isometric views of example tube and float systems.

FIGS. 2-5 show examples of different types of floats.

FIGS. 6A-6B show floats with chemical coatings.

FIGS. 7A-7B show an isometric view and a cross-sectional view along a line I-I, shown in FIG. 7A, respectively, of a float 700.

FIG. 8 shows an isometric view of an example tube and float system.

FIG. 9 shows an isometric view of an example tube and float system.

FIGS. 10A-10H show an example method of isolating and characterizing target materials of a suspension using a tube and float system.

FIGS. 11A-11B show an example method of isolating and characterizing target materials of a suspension using a tube and float system.

### DETAILED DESCRIPTION

A suspension is a fluid containing particles that are sufficiently large for sedimentation. A typical suspension may contain, in addition to a sought after target material, a wide variety of other materials. Examples of suspensions include blood, bone marrow, cystic fluid, ascites fluid, stool, semen, cerebrospinal fluid, nipple aspirate fluid, saliva, amniotic fluid, vaginal secretions, mucus membrane secretions, aqueous humor, vitreous humor, vomit, and any other physiological fluid or semi-solid. A target material can be cells, organisms, or particles whose density equilibrates when the suspension is centrifuged. Examples of target materials found in suspensions obtained from living organisms include cancer cells, inflammatory cells, viruses, parasites, and microorganisms, each of which has an associated specific gravity or density. When the suspension is added to a tube and float system and centrifuged, the various materials separate into different layers along the axial length of the tube according to their specific gravities. The float can be selected with a specific gravity to substantially match that of the target material. As a result, after centrifugation, the float is ideally positioned at approximately the same level as a layer containing the target material and expands the axial length of the layer containing the target material so that nearly the entire quantity of target material is positioned between the float outer surface and the inner surface of the tube. However, when a suspension contains at least one type of target material and other non-target materials having a similar density to that of the target material also fill the region between the outer surface of the float and the inner surface of the tube, isolation and characterization of the target material can be difficult.

Systems and methods described in this disclosure are directed to attaching the at least one target materials to the float and/or tube inner wall so that the target material can be isolated and reagents can be introduced to characterize the potentially different types of target materials based on molecular analysis or other observable properties exhibited by the target materials.

The detailed description is organized into two subsections as follows: Various tube and float systems for isolating and attaching target materials in a suspension are described below in a first subsection. Methods for characterizing the target materials using the tube and float systems are described in a second subsection.

### Tube and Float Systems

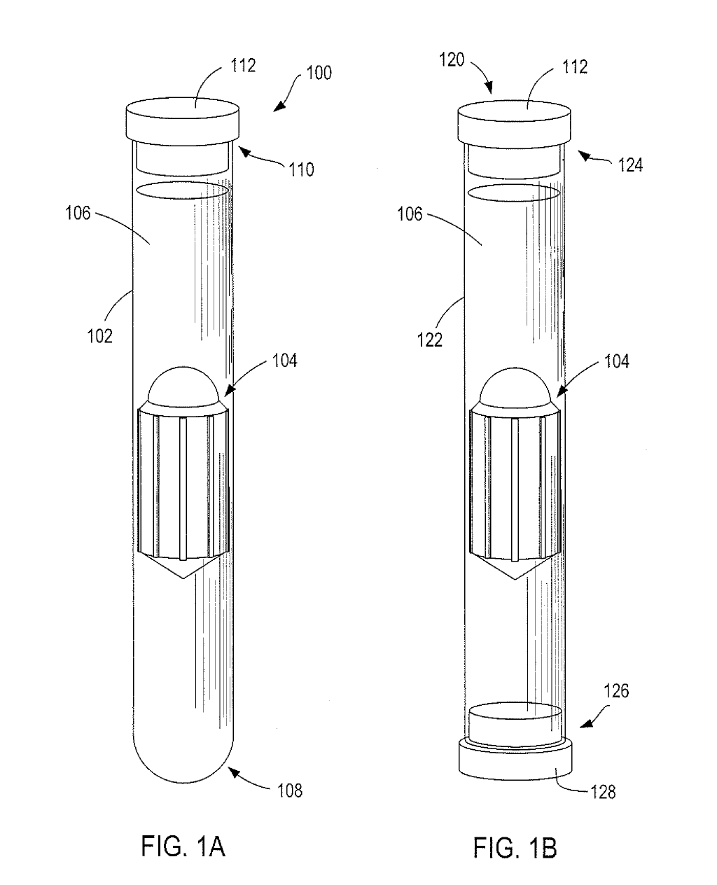

FIG. 1A shows an isometric view of an example tube and float system 100. The system 100 includes a tube 102 and a float 104 suspended within a suspension 106. In the example of FIG. 1A, the tube 102 has a circular cross-section, a first closed end 108, and a second open end 110. The open end 110 is sized to receive a stopper or cap 112, but the open end 110 can also have threads (not shown) to receive a threaded stopper or screw cap 112 that can be screwed onto the open end 110. FIG. 1B shows an isometric view of an example tube and float system 120. The system 120 is similar to the system 100 except the tube 102 is replaced by a tube 122 that includes two open ends 124 and 126 configured to receive the cap 112 and a cap 128, respectively. The tubes 102 and 122 have a generally cylindrical geometry, but may also have a tapered geometry that widens toward the open ends 110 and 124, respectively. Although the tubes 102 and 122 have a circular cross-section, in other embodiments, the tubes 102 and 122 can have elliptical, square, triangular, rectangular, octagonal, or any other suitable cross-sectional shape that substantially extends the length of the tube. The tubes 102 and 122 can be composed of a transparent or semitransparent flexible material, such as plastic or another suitable material.

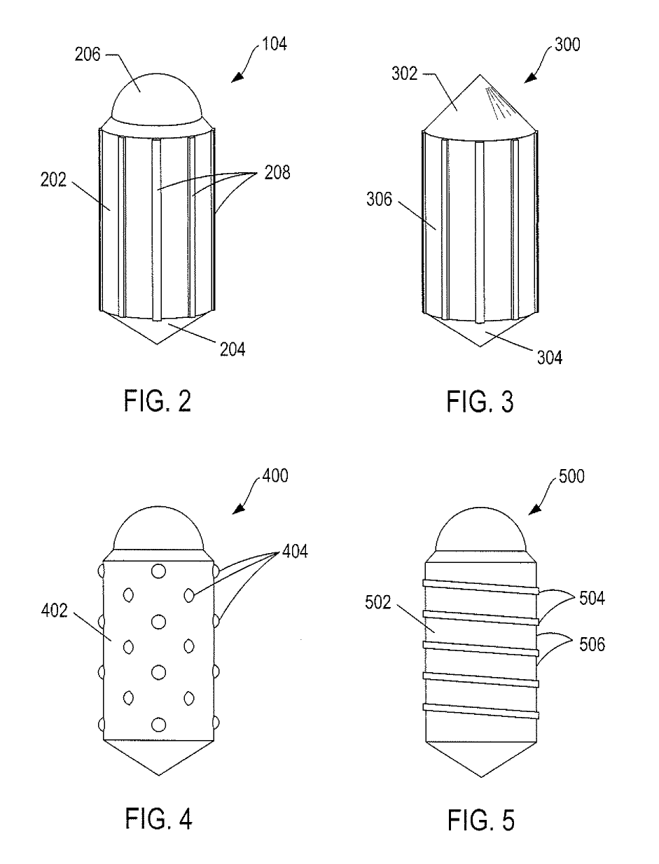

FIG. 2 shows an isometric view of the float 104 shown in FIG. 1. The float 104 includes a main body 202, a cone-shaped tapered end 204, a dome-shaped end 206, and splines 208 radially spaced and axially oriented on the main body 202. The splines 208 provide a sealing engagement with the inner wall of the tube 102. In alternative embodiments, the number of splines, spline spacing, and spline thickness can each be independently varied. The splines 208 can also be broken or segmented. The main body 202 is sized to have an outer diameter that is less than the inner diameter of the tube 102, thereby defining fluid retention channels between the outer surface of the body 202 and the inner wall of the tube 102. The surfaces of the main body 202 between the splines 208 can be flat, curved or have another suitable geometry. In the example of FIG. 2, the splines 208 and the main body 202 form a single structure.

Embodiments include other types of geometric shapes for float end caps. FIG. 3 shows an isometric view of an example float 300 with two cone-shaped end caps 302 and 304. The main body 306 of the float 300 includes the same structural elements (i.e., splines and structural elements) as the float 104. A float can also include two dome-shaped end caps.

In other embodiments, the main body of the float 104 can include a variety of different support structures for separating target materials, supporting the tube wall, or directing the suspension fluid around the float during centrifugation. FIGS. 4 and 5 show examples of two different types of main body structural elements. Embodiments are not intended to be limited to these two examples.

In FIG. 4, the main body 402 of a float 400 is similar to the float 104 except the main body 402 includes a number of protrusions 404 that provide support for the deformable tube. In alternative embodiments, the number and pattern of protrusions can be varied.

In FIG. 5, the main body 502 of a float 500 includes a single continuous helical structure or ridge 504 that spirals around the main body 502 creating a helical channel 506. In other embodiments, the helical ridge 504 can be rounded or broken or segmented to allow fluid to flow between adjacent turns of the helical ridge 504. In various embodiments, the helical ridge spacing and rib thickness can be independently varied.

A float can be composed of a variety of different materials including, but are not limited to, rigid organic or inorganic materials, and rigid plastic materials, such as polyoxymethylene (“Delrin®”), polystyrene, acrylonitrile butadiene styrene (“ABS”) copolymers, aromatic polycarbonates, aromatic polyesters, carboxymethylcellulose, ethyl cellulose, ethylene vinyl acetate copolymers, nylon, polyacetals, polyacetates, polyacrylonitrile and other nitrile resins, polyacrylonitrile-vinyl chloride copolymer, polyamides, aromatic polyamides (“aramids”), polyamide-imide, polyarylates, polyarylene oxides, polyarylene sulfides, polyarylsulfones, polybenzimidazole, polybutylene terephthalate, polycarbonates, polyester, polyester imides, polyether sulfones, polyetherimides, polyetherketones, polyetheretherketones, polyethylene terephthalate, polyimides, polymethacrylate, polyolefins (e.g., polyethylene, polypropylene), polyallomers, polyoxadiazole, polyparaxylene, polyphenylene oxides (PPO), modified PPOs, polystyrene, polysulfone, fluorine containing polymer such as polytetrafluoroethylene, polyurethane, polyvinyl acetate, polyvinyl alcohol, polyvinyl halides such as polyvinyl chloride, polyvinyl chloride-vinyl acetate copolymer, polyvinyl pyrrolidone, polyvinylidene chloride, specialty polymers, polystyrene, polycarbonate, polypropylene, acrylonitrite butadiene-styrene copolymer and others.

The surface of the main body of the float can be electrostatically charged so that attractive electrostatic forces attach target material particles to the surface of the main body of the float. Attractive electrostatic forces can be created by configuring the surface of the main body of the float with a net charge that is opposite the net charge of the target material particles. As a result, the target material attaches to the main body surface via attractive electrostatic forces.

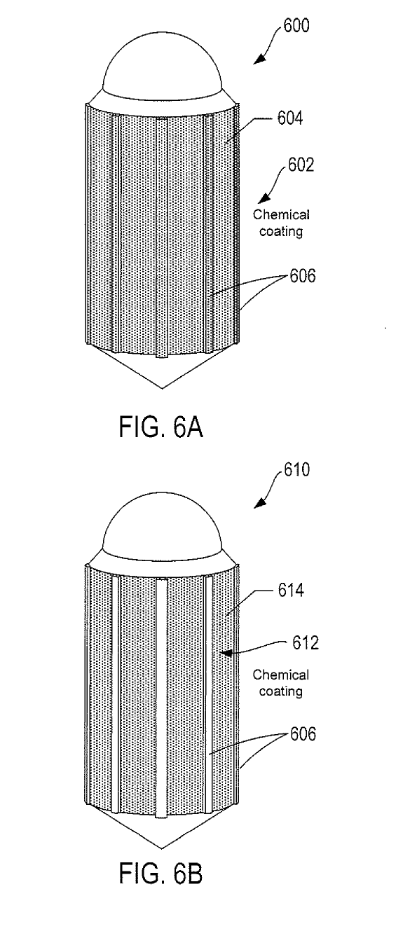

In certain embodiments, the surface of the main body of a float can be covered with a chemical layer that attaches or attracts the target material particles to the main body surface of the float. For example, the chemical layer can be a charged chemical layer or coating having a charge that is opposite the charge of the target material particles. Alternatively, the chemical coating can be a chemical attractant that causes the target material particles to migrate toward the main body surface, or the coating can be the surface of the main body of the float impregnated with a chemical attractant or adhesive. FIG. 6A shows a float 600 with a chemical coating represented by shaded surface 602 that covers the main body 604 and splines 606 of the float 600. The coating 602 is selected to enhance attachment of the target material particles to the main body 604 or causes the target material particles to migrate to the main body 604. FIG. 6B shows a float 610 with a chemical coating 612 that covers the main body 614 and not the splines 606 of the float 610. In certain example, the coatings 602 and 612 can be composed of a first material that possesses a net uniform negative charge to attach target material particles with a net positive charge. Alternatively, the coatings 602 and 612 can be composed of a second material that possesses a net uniform positive charge to attach target material particles with a net negative charge. For example, a typical circulating tumor cell (“CTC”) is a target material in anticoagulated whole blood with a net negative charge. In order to attach the CTCs to the main body of a float during centrifugation, the chemical coating can be a charged chemical coating with a net positive charge, such as ploy-D-lysine, that attaches the CTCs to the main body of the float. The chemical coating may also be a chemoattractant, such as epidermal growth factor or transforming growth factor alpha, tethered by antibodies that are attached to the main body 614. The chemoattractants cause CTCs to migrate in the direction of the main body 614.

Alternatively, in order to attach a variety of target materials of a suspension where certain target materials have a net positive surface charge and other target materials in the same suspension have a net negative charge, portions of the main body of a float can be covered with the first material to attach the target material particles with a net positive charge and other portions of the main body of the float can be covered with the second material to attach the target material particles with a net negative charge.

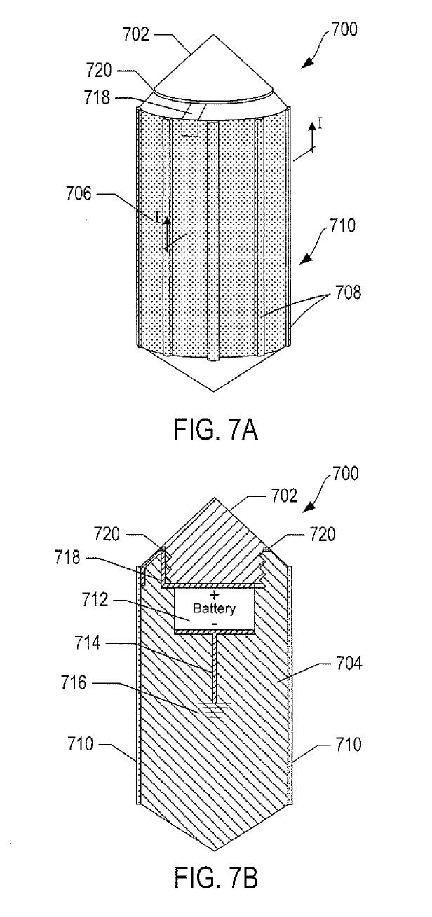

In other embodiments, the surface of the main body of a float can be covered with an electrically conductive coating and the float can include a battery that creates a charge in the coating to attach target material particles to the surface of the main body. FIGS. 7A-7B show an isometric view and a cross-sectional view along a line I-I, shown in FIG. 7A, respectively, of a float 700. The float 700 includes an insert 702 and a float exterior 704. As shown in FIG. 7A, the float exterior 704 includes a main body 706 and radially spaced splines 708. The main body 706 and splines 708 are covered with an electrically conductive coating 710. FIG. 7B reveals that the float 700 includes a cavity in which a battery 712 is inserted. The float 700 also includes a first electrode 714 with a first end in contact with the battery 712 and a second end in contact with a ground 716 and includes a second electrode 718 with a first end in contact with the battery 712 and a second end in contact with the electrically conductive coating 710, such as copper or aluminum. The ground 716 can be a piece of conductive metal, such as copper or aluminum, or the ground 716 can be the interior of the float exterior 704. In the example of FIG. 7B, the insert 702 and cavity are threaded so that the insert 702 can be screwed into the cavity with a gasket 720 disposed between the opening of the cavity and the insert 702. The coating 710 can be an electronically conductive polymer, or the coating 710 can be a transparent electronically conductive compound, such as indium tin oxide (“ITO”).

In the example shown in FIG. 7B, the battery 712 is inserted so that the positive terminal, denoted by “+,” contacts the second electrode 718 and the negative terminal, denoted by “−,” contacts the first electrode 714, giving the coating 710 a net positive charge. The float 700 can be used to attach target material particles with a net negative charge. For example, as described above, CTC's typically have a net negative surface charge and attach to the positively charged coating 710 during centrifugation. Alternatively, the battery 712 may be reversed, such that the positive terminal contacts the first electrode 714 and the negative terminal contacts the second electrode 718, so as to provide a net negative charge to the float 700. The float 700 may therefore be used to attach target material particles with a net positive charge.

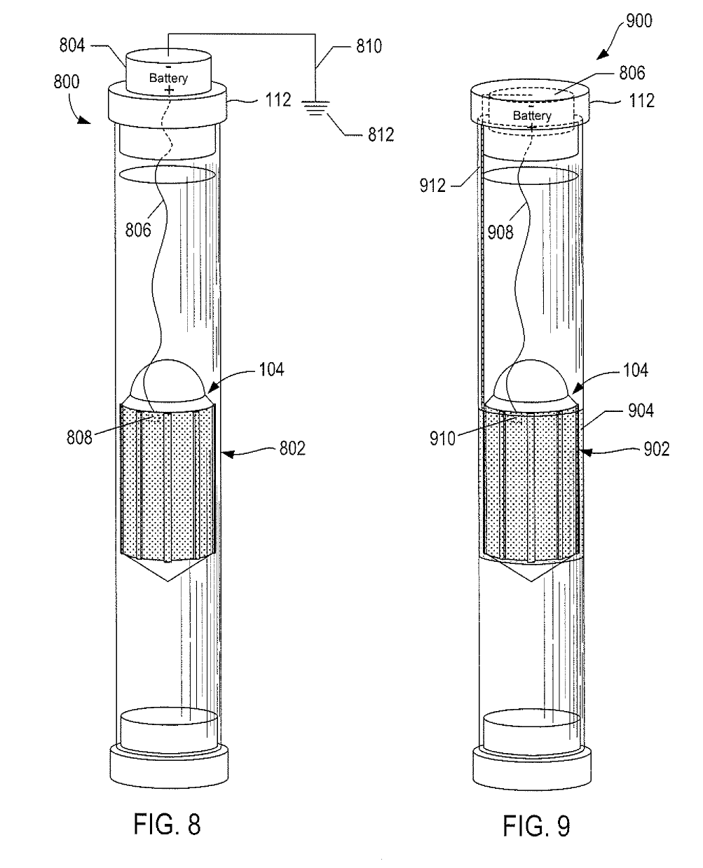

In still other embodiments, the battery can be disposed on, or embedded within, the cap of a tube and float system. FIG. 8 shows an isometric view of an example tube and float system 800. The system 800 is similar to the system 100 except the system 800 includes an electronically conductive coating 802 covering the main body of the float 104. The system 800 includes a battery 804 disposed on the cap 112, a first insulated wire 806 connected at a first end to the positive terminal “+” of the battery 804 and connected at a second end to a contact pad 808 disposed on the main body of the float 104 which, in turn, contacts the coating 802. The system 800 also includes a second insulated wire 810 connected at a first end to the negative terminal “−” of the battery 804 and connected at a second end to a ground 812. As shown in FIG. 8, a net positive charge is created in the coating 802, which enables target material particles with a net negative charge to attach to the coating 802 between the inner wall of the tube 102 and the float 104. Alternatively, the connections may be reversed, such that the positive terminal contacts the second insulated wire 810 and the negative terminal contacts the first insulated wire 806, so as to provide a net negative charge on the coating 802 to attract target material particles with a net positive charge to the coating 802 between the inner wall of the tube 102 and the float 104.

FIG. 9 shows an isometric view of the example tube and float system 900. The system 900 is similar to the system 100 except the system 900 includes a first electronically conductive coating 902 covering the main body of the float 104 and a second electronically conductive coating 904 covering the interior wall of tube 102. The system 900 includes a battery 906 embedded within the cap 112 and a first insulated wire 908 connected at a first end to the positive terminal “+” of the battery 906 and connected at a second end to a contact pad 910 disposed on the main body of the float 104 which, in turn, contacts the coating 902. The system 900 also includes a second insulated wire 912 connected at a first end to the negative terminal “−” of the battery 906 and connected at a second end to the second coating 904. The close proximity between the first and second coatings 902 and 904 creates a positive charge on the first coating 902 and a negative charge 904 on the second coating, enabling target material particles with a net negative charge to attach to the first coating 902 between the inner wall of the tube 102 and the main body of the float 104. Alternatively, the connections may be reversed so that a negative charge is created on the first coating 902 and a positive charge is created on the second coating 904, enabling target material particles with a net positive charge to attach to the first coating 902 between the inner wall of the tube 102 and the main body of the float 104.

A battery may also be connected to a high voltage amplifier to increase the charge. Because there is no flow of current, a higher potential can be achieved with a battery having a smaller potential.

### Methods for Characterizing Target Materials of a Suspension

For the sake of convenience, methods for characterizing a target material in a suspension are described with reference to an example suspension and example target material. In this example, the target materials are CTCs and the suspension is anticoagulated whole blood. Note however that methods disclosed herein are not intended to be so limited in their scope of application. The methods described below can, in practice, be generalized to isolate and characterize any kind of target material in nearly any kind of suspension and are not intended to be limited to isolating and characterizing CTCs of a whole blood sample.

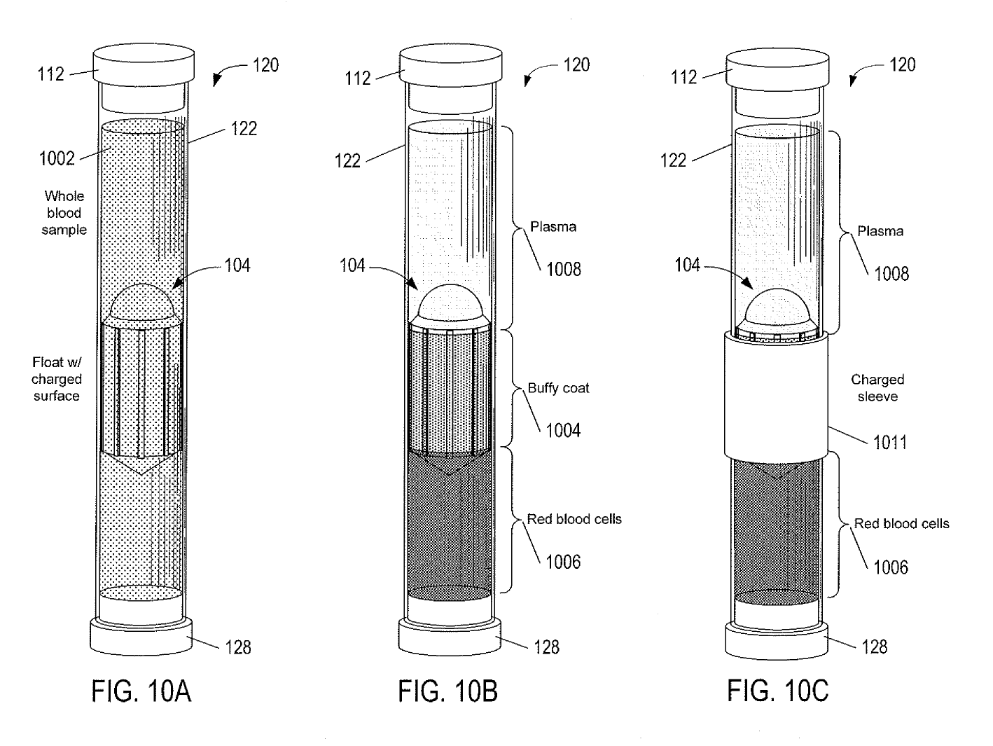

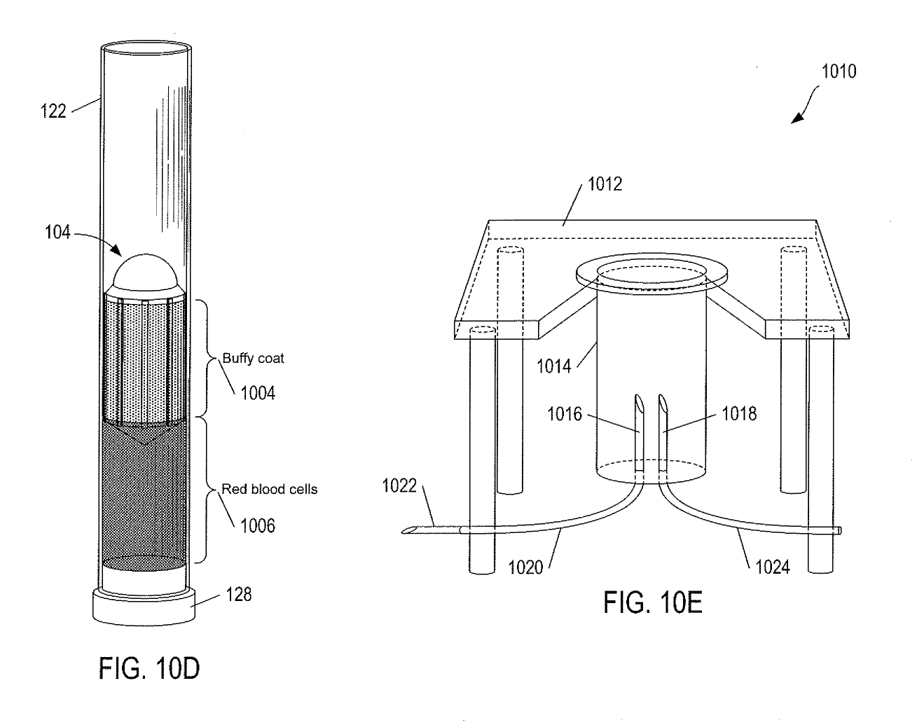

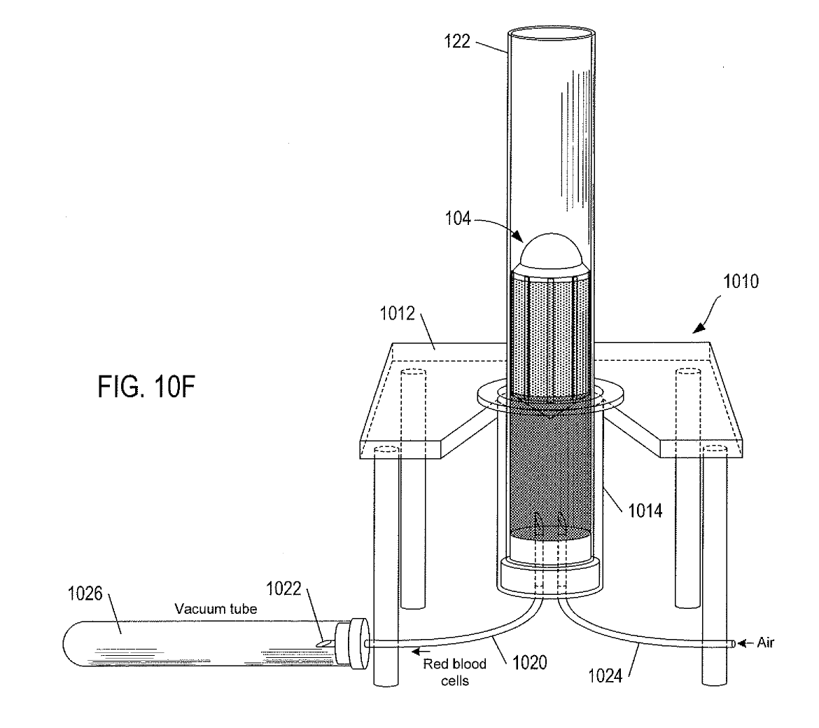

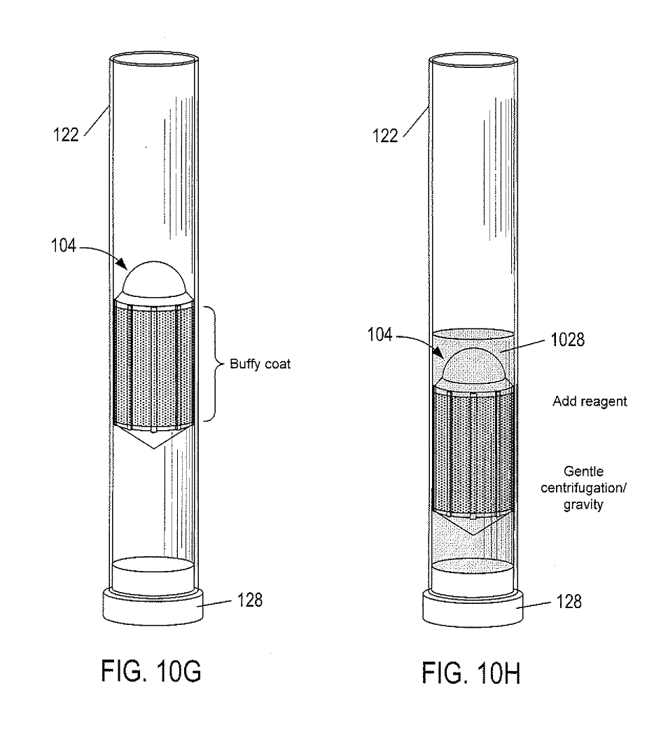

FIG. 10A shows an example of the tube and float system 120 filled with an anticoagulated whole blood sample 1002. The whole blood sample 1002 can be drawn into the tube 122 using venipuncture or by transferring the whole blood sample 1002 from a collection vessel, such as a vacuum tube, to the tube 122. Prior to drawing the whole blood sample into the tube 122, the float 104 is selected to have a specific gravity that positions the float 104 at approximately the same level as the buffy coat. The float 104 also includes a net positively charged main body surface to attach CTCs. In certain examples, the charged main body surface can be formed by coating the main body surface with a positively charged chemical coating, such as poly-D-lysine, poly-L-lysine, Cell-Tak™ adhesive, or a chemical attractant as described above with reference to FIG. 6. Alternatively, the float 104 can include a battery and the main body surface covered with an electronically conductive coating, as described above with reference to FIG. 7. The float 104 can then be inserted into the tube 122 followed by drawing the whole blood sample 1002 into the tube 122, or the float 104 can be inserted after the whole blood sample 1002 has been drawn into the tube 122. Because the presence of white blood cells (“WBCs”) can make the detection of CTCs trapped between the float 104 and inner wall of the tube 122 difficult, WBC antibodies may also be added to the blood sample to cause red blood cells (“RBCs”) to bind to the WBCs, thereby forming a WBC-RBC complex and increasing the specific gravity of the WBC-RBC complex. In the example shown in FIG. 10A, the cap 112 is inserted into the open end 124 of the tube 122.

The tube 122, float 104, and whole blood sample 1002 are centrifuged for a period of time sufficient to separate the particles suspended in the whole blood sample 1002 according to their specific gravities. FIG. 10B shows an example of the tube and float system 100 where the float 104 traps and spreads a buffy coat 1004 between a layer of packed red blood cells 1006 and plasma 1008. The centrifuged blood sample may actually be composed of six layers: (1) packed red cells 1006, (2) reticulocytes, (3) granulocytes, (4) lymphocytes/monocytes, (5) platelets, and (6) plasma 1008. The reticulocyte, granulocyte, lymphocytes/monocyte, platelet layers form the buffy coat 1004 and are the layers often analyzed to detect certain abnormalities, such as CTCs. In FIG. 10B, the float 104 is positioned to expand the buffy coat, enabling the negatively charged CTCs to attach to the positively charged coated main body surface of the float 104. In FIG. 10C, in order to increase the likelihood that the CTCs contact the main body of the float 104, the tube 122 can be inserted in an appropriately charged sleeve 1011. For example, the sleeve 1011 can be negatively charged in order to repel the negatively charged CTCs away from the tube 122 inner wall toward the main body of the float 104. If WBC antibodies have been added to the blood sample prior to centrifugation, the higher density WBC-RBC antibody complexes are within the packed red blood cells 1006 beneath the float 104.

If CTCs are present, they may be identified through the tube 122 wall. On the one hand, if no CTCs are detected between the float 104 outer surface and the inner wall of the tube 122, or if no significant change in the number and characterization of the CTCs is detected since the last test, no further processing is required and the method stops. On the other hand, if CTCs are detected and characterization of the CTC's is desired, the cap 112 can be removed and the plasma 1008 and buffy coat 1004 can be poured off or aspirated with a pipette. FIG. 10D shows the plasma 1008 and buffy coat 1004 removed from the tube 102. The negatively charged CTCs are attached to the positively charge coating covering the main body of the float 104.

FIG. 10E shows a system 1010 for extracting the red blood cell 1006. The system 1010 includes a stand 1012 configured to receive a translucent tube holder 1014. The holder 1014 has an open end dimensioned to receive the tube 122 and cap 128, and two hypodermic needles 1016 and 1018 directed into the cavity of the holder 1014. The needle 1016 is connected to a first end to a flexible tube 1020, which is connected at a second end to a needle 1022. The needle 1018 is also connected to a flexible tube 1024.

As shown in FIG. 10F, the tube 122 and cap 128 are inserted into the cavity of the holder 1014 so that needles 1016 and 1018 puncture the cap 128. The cap 128 can be composed of rubber or include a rubber region through which the needles can puncture to form a liquid tight seal around the needles 1016 and 1018. The needle 1022 is then inserted into a vacuum tube 1026. The red blood cells and other materials and fluids trapped below the float 104 are sucked through the tube 1020 and into the vacuum tube 1026 and air is drawn into the volume of the tube 122 beneath the float 104 to release back pressure. Alternatively, the vacuum tube 1026 may be a vacuum trap connected to a vacuum system or a pump system.

In alternative embodiments, because the target materials are attached to the main body of the float 104 and when the float 104 with protrusions, a helical rib, or splines is used, the second needle 1018 and tube 1024 can be omitted from the system 1010 and air to release back pressure can be drawn into the region beneath the float 104 via the channels between the main body of the float 104 and the inner wall of the tube 122.

FIG. 10G shows the tube 122 and cap 128 removed from the holder 1014 with the red blood cells and other fluids removed.

In the event that any residual materials are not removed when the plasma 1008, buffy coat 1004, and red blood cells 1006 are removed, a wash 1028, such as saline solution or another suitable reagent, can be introduced to the tube 122, as shown in FIG. 10H. The tube 122, float 104, and wash 1014 can be gently centrifuged, or the wash 1028 can be allowed to settle via gravity in the channels to suspend any residual material. The tube 102 can also be expanded by applying air pressure within the tube 102, by exerting a force on a top or bottom portion of the tube 102, or by introducing a vacuum by inserting the tube 102 into an adapter and removing the pressure between the tube 102 and the adapter to allow the wash 1028 to enter the channels. The wash 1028 can be aspirated or drained using the system 1010, as described above with reference to FIG. 10F.

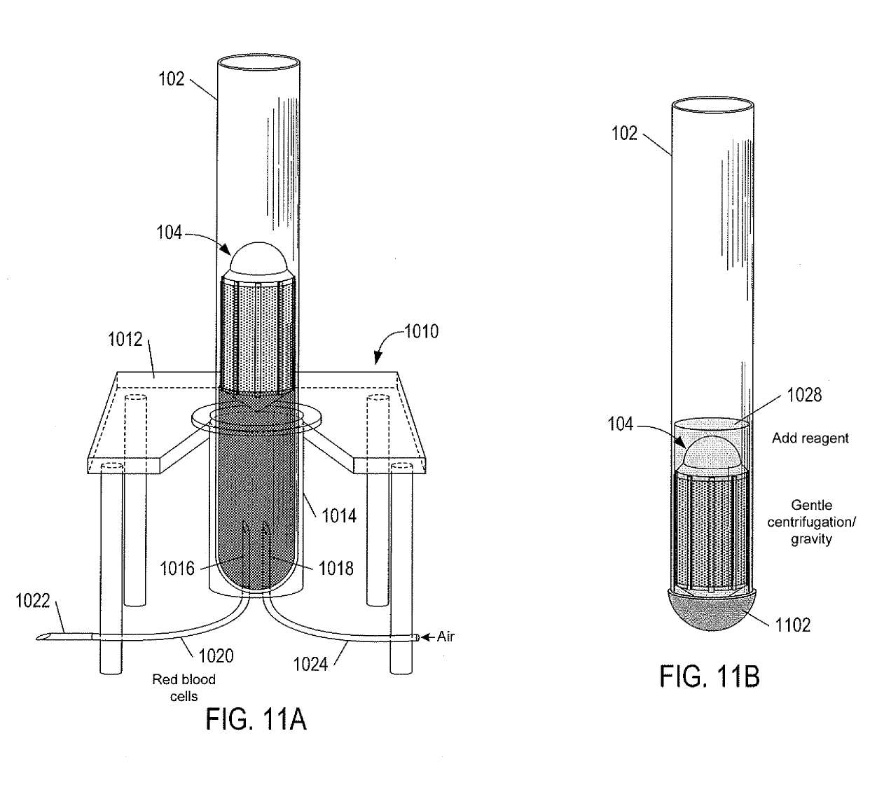

The same procedure described above with reference to FIGS. 10A-10H can be used isolate target materials attached to the main body of the float 104 of the tube and float system 100. FIG. 11A shows the tube 102 inserted into the cavity of the holder 1014 so that needles 1016 and 1018 puncture the closed end 108 of the tube 102. The red blood cells and other materials can be drawn off from beneath the float by attaching a vacuum tube, as described above with reference to FIG. 10F.

As shown in FIG. 11B, a cap 1102 can be placed over the bottom of the tube 102 to cover the holes 1102 and 1104 and the wash 1028 can be introduced to the tube 102.

After the reagent is introduced, the CTCs can be incubated on the float 104 in the tube for a period of time and characterized. Note that washing and introducing reagents can be repeated for subsequent rounds of incubation.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Obviously, many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the following claims and their equivalents:

1. A system for isolating and characterizing a target material of a suspension, the system comprising:
a float with a main body; and
a tube with at least one opening to receive the float and the suspension, the float to create forces that attract the target material particles into a region of space between the main body and inner wall of the tube.

2. The system of claim 1, wherein the float further comprises:
an insert;
a float exterior with a cavity to receive a battery;
an electrically conductive coating disposed on at least a portion of the main body;
a first electrode connected at a first end to the battery and connected at a second end to the electrically conductive coating; and
a second electrode connected at a first end to the battery and connected at a second end to a ground.

3. The system of claim 2, wherein the electrically conductive coating further comprises indium tin oxide.

4. The system of claim 2, wherein the electrically conductive coating further comprises a conductive polymer.

5. A method for harvesting at least one target material of a suspension, the method comprising:
centrifuging the suspension in a tube and float system, wherein the electrostatically charged main body attaches target material particles to the main body of the float;
removing non-target material layers from the tube;
introducing a reagent to characterize the target material particles attached to the main body of the float;
incubating the target material particles on the float and in the tube for a period of time; and
characterizing the target material attached to the main body of the float.

6. The method of claim 5, wherein removing the non-target material layers further comprises removing the layers of above the float with a pipette.

7. The method of claim 5, wherein removing the non-target material layers further comprises:
inserting a needle connected to a vacuum/containment device into the tube; and
vacuuming the non-target material from beneath the float.

8. The method of claim 5 further comprising introducing a wash to remove any residual non-target materials.
